(19) United States
(12) Patent Application Publication
(10) Pub. No.: US 2012/0015044 A1
(43) Pub. Date: Jan. 19, 2012

(54) FORMULATIONS FOR THE TREATMENT OF URUSHIOL-INDUCED CONTACT DERMATITIS

(75) Inventors: Laszlo TREIBER, San Diego, CA (US); Angela HANSEN, San Diego, CA (US)

(73) Assignee: Laszlo TREIBER, San Diego, CA (US)

(21) Appl. No.: 13/180,485

(22) Filed: Jul. 11, 2011

Related U.S. Application Data
(60) Provisional application No. 61/399,507, filed on Jul. 13, 2010.

(51) Int. Cl. A61K 33/40; A61P 17/00

(52) U.S. Cl. 424/616

(57) ABSTRACT

Disclosed herein are compositions comprising: a pharmaceutically acceptable oxidizing agent; a pharmaceutically acceptable base; and a pharmaceutically acceptable solvent or mixture of solvents. Also disclosed are methods of treating urushiol-induced contact dermatitis in a patient, the method comprising: identifying a patient in need thereof; and administering to the patient a composition as described herein. Further disclosed herein are devices for the treatment of urushiol-induced contact dermatitis, the device comprising: an absorbent material, contained therein a composition as described herein; and means for attaching the device to the skin of a patient in need thereof.

### RELATED APPLICATION

The present application claims priority to the U.S. Provisional Application Ser. No. 61/399,507, filed on Jul. 13, 2010 by Laszlo Treiber, et al., and entitled “FORMULATIONS FOR THE TREATMENT OF URUSHIOL-INDUCED CONTACT DERMATITIS,” the entire disclosure of which is incorporated herein by reference.

### FIELD OF THE INVENTION

The present invention is in the field of lotions and creams, and in particular formulations for the treatment of urushiol-induced contact dermatitis.

### BACKGROUND OF THE DISCLOSURE

An estimated 70-85% of the North American population will develop the allergic reaction known as “contact dermatitis” when exposed to either of the poisonous plants known as poison ivy, poison oak and poison sumac, whose sap contains urushiol, the allergen responsible for the reaction. Annually, millions of people in the U.S. alone develop the condition known as urushiol-induced contact dermatitis (UICD). The symptoms of UCID are rash and blistering on the contacted areas of skin accompanied by severe burning and itching, which may last 2-3 weeks. In those that are very sensitive to urushiol, the rash may take up to 5-6 weeks to heal. Many cases require medical attention.

The group of allergens responsible for the allergic reaction consists of catechol derivatives substituted on the 3-position that is known as urushiol, which is a compound of Formula I:

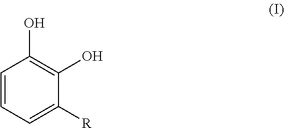

where R is an alkane or alkene chain of 15 carbons long, for example, (CH₂)₁₄CH₃; (CH₂)₇CH═CH(CH₂)₅CH₃; (CH₂)₇CH═CHCH₂CH═CH(CH₂)₂CH₃; (CH₂)₇CH═CHCH₂CH═CHCH═CHCH₃; (CH₂)₇CH═CHCH₂CH═CHCH₂CH═CH₂; and the like.

Current treatments for poisonous plant contact include physical removal of the sap from the skin with cleansing agents such as soap and water, rubbing alcohol, lotions containing polymer particles, etc. However, these forms of treatment can only be successful, if the exposure is noticed within a short time (no more than 10 minutes) following the contact with the plant. Once urushiol, acting as a hapten, is chemically bound to skin proteins forming a molecular entity called a hapten adduct, or a hapten-carrier adduct, it cannot be removed by physical methods such as washing, cleansing and scrubbing. The current standard of care recommended by medical associations for self-treatment, once the symptoms of rash and itch are present, include antihistamines, calamine lotion and over-the-counter cortisone creams. The more serious cases are treated with prescriptions of oral corticosteroids. It is important to note, that all of these methods of treatment are limited to addressing the symptoms by reducing the burning and itching sensation, but they do not eliminate the cause of the allergic reaction. Therefore, they do not shorten the course of recovery.

Therefore, a need exists in the art for a safe, easy, and effective treatment for contact with poisonous plants.

### SUMMARY OF THE INVENTION

Disclosed herein are compositions comprising: a pharmaceutically acceptable oxidizing agent; a pharmaceutically acceptable base; and a pharmaceutically acceptable solvent. Also disclosed are methods of treating urushiol-induced contact dermatitis in a patient, the method comprising: identifying a patient in need thereof; and administering to the patient a composition as described herein. Further disclosed herein are devices for the treatment of urushiol-induced contact dermatitis, the device comprising: an absorbent material, contained therein a composition as described herein; and means for attaching the device to the skin of a patient in need thereof.

### DETAILED DESCRIPTION OF THE EMBODIMENTS

Once urushiol has penetrated the skin, it is very difficult to remove it by simple washing techniques. This is especially true when urushiol binds to skin proteins, or other biological molecules, and forms hapten adducts. Urushiol is a catechol derivative, and as such is susceptible to oxidation. The resulting oxidized catechol moiety does not form an allergen with biological molecules. Furthermore, urushiol already bound in a protein complex is also oxidized under the same conditions to form the oxidized product that is no longer bioactive and can be removed from the skin relatively easily. Therefore, the present inventors have discovered that if urushiol is oxidized in situ after the exposure to the skin, UICD can be effectively treated.

Thus, in one aspect, disclosed herein are compositions comprising:
a pharmaceutically acceptable oxidant;
a pharmaceutically acceptable base; and
a pharmaceutically acceptable solvent.

A “pharmaceutically acceptable” compound or mixture is one which when applied to an organism, such as a mammal, does not cause an adverse reaction to the extent that makes such application undesirable. The term “pharmaceutically acceptable,” therefore, is a relative term. For instance, if the compound is being applied to treat a rash, but the compound itself causes rash to develop, if subsequent to the application of the compound the overall extent of rash is reduced, then the compound is pharmaceutically acceptable.

In some embodiments, the oxidant is a peroxide derivative. Peroxide derivatives are compounds having the dioxygen —O—O— linkage in the compound. Peroxides fall within several categories. Examples include an organic peroxide (R₁—O—O—R₂, where R₁ and R₂ are hydrogen or an organic group), organic hydroperoxide (R₁—O—O—H), or a peracid or perester (R₁—C(═O)O—O—R₂, where R₁ and R₂ are hydrogen or an organic group). While some compounds within each of the above categories are suitable for use with the compositions disclosed herein, in some embodiments, the peroxide is a compound of formula R—O—O—H, where R is selected from the group consisting of hydrogen, optionally substituted alkyl, acyl, and optionally substituted aryl. In some embodiments, the peroxide is selected from the group consisting of hydrogen peroxide, hydrogen peroxide urea adduct and benzoyl peroxide. In some embodiments, the peroxide is in a pharmaceutically acceptable concentration, i.e., a concentration that is high enough to oxidize the catechol but is low enough that does not cause the oxidation of the patient's tissue to an undesirable extent. An example of a pharmaceutically acceptable concentration of a peroxide is hydrogen peroxide having a concentration of up to 3% w/w.

In some embodiments, the oxidizing agent is oxygen. In some of these embodiments, exposing the affected skin area to the base and the solvent without covering the skin enhances the effect of oxygen present in the air to oxidize the urushiol. In other embodiments, the composition comprising the base and the solvent is aerated or oxygenated, such that the concentration of dissolved oxygen molecules in the compositions is greater than the concentration of naturally occurring oxygen in the mixture. In some embodiments, the composition where the oxidizing agent is oxygen has high pH, i.e., pH greater than 10.

In some embodiments, the base is an inorganic base. Examples of inorganic bases include ammonia; hydroxides; phosphates, such as phosphate, hydrogen phosphate, carbonate, bicarbonate, and the like. In other embodiments, the base is an organic base. Examples of organic bases include amines, primary amines, secondary amines, tertiary amines, carboxylates, and the like. In some of these embodiments, the organic base is an organic amine.

In some embodiments, the pharmaceutically acceptable base is in the form of a buffer mixture. In some embodiments, the buffer mixture is a buffer solution. In some embodiments, the buffer is an aqueous buffer. In some of these embodiments, the buffer is in the pH range of about 8.5 to about 12. Throughout the present disclosure the term “about” a certain value means that a range of value±10%, and preferably a range of value±5%, is contemplated. Thus, for example, a pH of about 12 includes a pH in the range of 10.8 and 13.2, or pH in the range of 11.4 and 12.6.

In some embodiments, the buffer is selected from the group consisting of bicarbonate buffer, carbonate buffer, phosphate buffer, borax, acetate buffer, 2-bis(2-hydroxyethyl)amino-2-(hydroxymethyl)-1,3-propanediol (BIS-TRIS), 1,3-Bis[tris(hydroxymethyl)methylamino]propane (BIS-TRIS Propane), N-(2-hydroxyethyl)piperazine-N′-(2-ethanesulfonic acid) sodium salt (HEPES sodium salt), 4-morpholineethanesulfonic acid sodium salt (MES Sodium Salt), 4-morpholinepropanesulfonic acid sodium salt (MOPS Sodium Salt), and tris(hydroxymethyl)aminomethane (Tris Base; Trizma®). Other buffers used in medical, biological, or biochemical arts can be used with the present compositions.

In some embodiments, the organic amine is a compound of formula R—NH₂, wherein R is selected from the group consisting of hydrogen, optionally substituted alkyl, and optionally substituted aryl. In certain embodiments, the organic amine is water soluble. In some embodiments, the organic amine is selected from the group consisting of triethanolamine, tris(hydroxymethyl)aminomethane, and biologically acceptable esters of naturally occurring amino acids.

In some embodiments, the disclosed compositions further comprise a pharmaceutically acceptable transdermal transport agent. Transdermal transport agents are those that carry organic compounds across the skin and allow for the skin penetration of an otherwise impenetrable compound. In some embodiments, the transdermal transport agent operates through an intercellular pathway, whereas in other embodiments, the agent operates through an intrafollicular pathway.

In some embodiments, the transdermal transport agent is a solvent that enhances penetration through both polar and non-polar pathways. Examples of such solvents include, but are not limited to, 2-pyrrolidone, N-methyl pyrrolidone, N-methyl formamide, and propylene glycol, all of which can optionally be applied in combination with laurocapram. In other embodiments, the transdermal transport agent preferentially affects the polar route. An example of such enhancers includes, but are not limited to, propylene glycol in combination with decylmethylsulfoxide. In other embodiments, the transdermal transport agent preferentially affects the non-polar route. Examples of such enhancers include, but are not limited to, propylene glycol in combination with oleic acid, propylene glycol alone, and water.

Some transdermal transport agents are relatively simple organic compounds, such as ethyl alcohol, isopropyl alcohol, propylene glycol, acetone, 2-pyrrolidone, laurocapram (Azone), isopropyl myristate, oleic acid, and dimethyl sulfoxide, and combinations thereof. Other transdermal transport agents have more complex structures, for example a phospholipid liposome (see, for example Sen et al., Biophys J. 2002, 83(4):2064-2073), or a lecithin organogel (see, for example, Walde et al., J Pharm Sci. 1992, 81(9):871-4). Some of the mechanisms of skin penetration and examples of compounds that affect the same are reviewed in Nava Dayan, “Pathways for Skin Penetration,” Cosmetics & Toiletries, vol. 120, no. 6, pp. 67-76 (June 2005) (www.TheCosmeticSite.com), the entire disclosure of which is incorporated by reference herein, especially the subject matter related to skin penetrants and enhancers.

In some embodiments, the organic amine and the peroxide are soluble in the transdermal transport agent, or can be encapsulated in the transdermal transport agent if the agent is a liposome.

In some embodiments, the pharmaceutically acceptable solvent is an aqueous solvent, comprising mainly of hydrophilic components. In some embodiments, the solvent is a non-aqueous solvent, comprising mainly of hydrophobic components. In some embodiments, the solvent used in the compositions disclosed herein is a mixture of solvents. In some embodiments, one of the solvents used is water. As described below, in some embodiments, the solvent comprises components that cause the formulation to form a gel, cream, or foam.

In some embodiments, the compositions disclosed herein further comprise abrasive particles. The abrasive particles help break the outer layer of skin and allow for a more efficient penetration of the composition into the skin. The presence of the abrasive particles can increase the effective concentration of the active compounds, i.e., more compound penetrates the skin, and decrease the amount of time it takes for the maximum concentration to reach the affected area. In some embodiments, the abrasive particle is selected from the group consisting of salt (NaCl), ground nut shell, ground sand, various insoluble ground polymers, and ground pumice.

The compositions disclosed herein can be formulated in a number of different ways for application to the skin. In some embodiments, the compositions are in aqueous liquid form, where the composition is simply applied to the skin. In some of these embodiments, the composition is in a spray bottle and it is sprayed onto the affected areas. In other embodiments, the composition is poured out of a spout on the bottle and, optionally, is rubbed onto the skin.

In other embodiments, the composition is formulated in the form of a gel or cream. Various gel, suspension, or cream formulations for the application of water soluble material are known in the art, for example in the cosmetic arts. The compositions disclosed herein can be formulated in the form of an ointment or hand lotion, or the like.

In yet other embodiments, the compositions disclosed herein are formulated in the form of a foam. The composition is held in a bottle under some pressure and when the composition is released, it foams and the foam is then applied to the affected area. Various foam formulations for the application of water soluble material are known in the art.

By “solution” it is meant that all of the components in the mixture are uniformly distributed throughout the solvent such that there is a molecular interaction between the solvent molecules and the solute molecules. A “suspension,” on the other hand, refers to a mixture where the components within the mixture form aggregates, where aggregates are uniformly distributed throughout the mixture. A suspension can be an emulsion, as that term is understood in the art.

In another aspect, the compositions disclosed herein are pre-applied to an applicator and the applicator is packaged for sale. In these embodiments, the applicator is removed from its packaging and then is applied to the affected skin area, either by rubbing the applicator against the affected area, or by attaching or sticking the applicator over the affected area. Some examples include: a patch that sticks to the affected area and slowly leaches the disclosed composition onto the skin; an absorbent material, such as gauze or cotton, that is soaked in the composition, analogous to rubbing alcohol pads.

Thus, disclosed herein are devices for the treatment of urushiol-induced contact dermatitis, the device comprising:
an absorbent material, contained therein a composition as discussed above; means for attaching the device to the skin of a patient in need thereof.

In some embodiments, the device is a relatively flat absorbent material, for example of a defined geometric shape, such as a circle or a square or the like, where the edges of the device comprise an adhesive material. When the device is applied to the skin, the adhesive material causes the device to adhere to the skin. The absorbent material in the device then slowly releases the disclosed composition onto the skin.

In other embodiments, the device comprises adhesive bands that can cause the device to adhere to the skin. In further embodiments, the device is attached to the skin using pieces of tape.

In another aspect, disclosed herein are methods of treating urushiol-induced contact dermatitis in a patient, the method comprising:
identifying a patient in need thereof; and
administering to the patient a composition as described above.

In some embodiments, the administering step comprises rubbing the composition on the skin of the patient, while in other embodiments the administering step comprises placing an absorbent material comprising the composition on the skin of the patient.

In some embodiments, the oxidizing agent and the remaining ingredients are not chemically compatible. In these embodiments, the oxidizing agent slowly oxidizes at least one of the other ingredients, thereby reducing the efficacy of the composition. The shelf-life of the pre-mixed, single mixture, then, would be rather short, i.e., not sufficient for effective commercialization of the product. In these embodiments, then, the composition cannot be packaged and sold as a single mixture. Instead, the composition needs to be mixed shortly prior to usage, for example less than 15 minutes before usage, or less than 5 minutes before usage.

Thus, in some embodiments, the packaging is a single chamber packaging, which contains the composition as disclosed herein. In other embodiments, the packaging is a multichamber packaging, where each chamber contains a separate mixture or solution. In some embodiments, one chamber holds a mixture or solution comprising the oxidizing agent. Another chamber holds a mixture or solution comprising the additional ingredients. In other embodiments, one chamber holds a mixture of the oxidizing agent and one or more other ingredient that is inert towards oxidization by the oxidizing agent.

Other packaging configurations contemplated include, but are not limited to: separate bottles to hold liquids, tube-within-a-tube for gels, double barrel syringes, dual chambered pumps, single use packets, and the like.

The formulations described herein chemically alter the allergen (compound of Formula I) in its free form, as well as after its attachment to skin proteins to form a hapten adduct, thereby eliminating the cause of the reaction in all stages of the exposure from the initial contact through the full allergic reaction.

As demonstrated by in vitro laboratory experiments, the key chemical reaction is the oxidative conversion of the catechol ring resulting in non-allergenic derivatives. Clinical case studies of UICD also suggest the efficacy of the formulations' mechanism of action as the subjects reported substantial relief from symptomatic burning and itching for a few hours post each treatment and complete recovery within 2-3 days after the initiation treatment series.

The compositions disclosed herein take advantage of the sensitivity of the catechol ring toward oxidizing agents at high pH. The preferred pH range is 8.5-12, where the rate of oxidation of the catechol ring is adequate without any danger of damage to the skin. The choice of oxidizer can be any of the biologically acceptable oxidizing agents such as peroxide derivatives, preferably hydrogen peroxide. The high pH is established by using a biologically or pharmaceutically acceptable inorganic or organic base or buffer, preferably an organic base with the ability to penetrate the skin.

The significance of the formulations described in this invention is the ability to oxidize and thus chemically remove urushiol in the bound (hapten) and unbound state. In this way, the formulations' activity addresses both the symptoms and the actual cause of the allergic reaction, thereby working to decrease the reaction's intensity and time until resolution. This illustrates the efficacy, practicality the immense demonstrated benefit of this invention, when compared to other methods of treatment that focus either on the physical removal of the unbound urushiol (successful only for a narrow window of time) or on simply the symptomatic treatment once the dermatitis has developed (which do not affect the length of the reaction time).

### EXAMPLES

### Example 1

Solution A: NaHCO₃ (sodium bicarbonate) dissolved in water to obtain a saturated solution of ca. 8% (w/w).

Solution B: 7 parts of 70% or higher isopropyl alcohol known as “rubbing alcohol” and 3 parts of 3% (w/w) hydrogen peroxide (H₂O₂ in water) were mixed.

Freshly harvested leaves of poison oak were minced and rubbed into the selected skin surface of a volunteer subject. This exposure represented an overexposure relative to accidental and unintentional contact with the poisonous plant. After approximately 24 hours the symptoms of UICD were confirmed and the treatment was started. Immediately before application to the affected skin of the volunteer, Solution A (1 part) was added to Solution B (9 parts) and thoroughly mixed. An appropriate carrier (i.e., a paper towel) was saturated with the resulting mixture and held tightly to the area showing the symptoms of dermatitis for 20-30 min. (Other carriers, e.g. cotton tissue, gauze, etc., can also be used.) As a result of treatment, discomfort such as burning and itching rapidly subsided and remained tolerable for a few hours. Upon the return of burning and itching the treatment was repeated. As a result of periodic treatments as needed, the symptoms were permanently eliminated typically within 36-48 hours following the treatment initiation (depending on the sensitivity of the individual and the extent of the exposure to the poisonous plant).

### Example 2

Solution A: Na₂CO₃ was dissolved in water to obtain a 10% (w/w) solution.

Solution B: 9 parts of isopropyl alcohol and 1 part of 25-30% (w/w) hydrogen peroxide (H₂O₂ in water) were mixed.

Freshly harvested leaves of poison oak were minced and rubbed into the selected skin surface of a volunteer subject. This exposure represented an overexposure relative to accidental and unintentional contact with the poisonous plant. After approximately 24 hours the symptoms of UICD were confirmed and the treatment was started. Immediately before application to the affected skin of the volunteer, Solution A (1 part) was added to Solution B (9 parts) and thoroughly mixed. An appropriate carrier (i.e., a paper towel) was saturated with the resulting mixture and held tightly to the area showing the symptoms of dermatitis for 20-30 min. (Other carriers, e.g. cotton tissue, gauze, etc., can also be used.) As a result of treatment, discomfort such as burning and itching rapidly subsided and remained tolerable for a few hours. Upon the return of burning and itching, the treatment was repeated. As a result of periodic treatments as needed, the symptoms were permanently eliminated typically within 36-48 hours following treatment initiation (depending on the sensitivity of the individual and the extent of the exposure to the poisonous plant).

### Example 3

Solution A: triethanolamine was dissolved in 70% v/v or higher isopropyl alcohol (“rubbing alcohol”) to obtain a 3% solution.

Solution B: 30% (w/w) hydrogen peroxide (H₂O₂ in water).

Freshly harvested leaves of poison oak were minced and rubbed into the selected skin surface of a volunteer subject. This exposure represented an overexposure relative to accidental and unintentional contact with the poisonous plant. After approximately 24 hours the symptoms of UICD were confirmed and the treatment was started. Immediately before application to the affected skin of the volunteer, Solution B (1 part) was added to Solution A. (9 parts) and thoroughly mixed. An appropriate carrier (i.e., a paper towel) was saturated with the resulting mixture and held tightly to the area showing the symptoms of dermatitis for 20-30 min. (Other carriers, e.g. cotton tissue, gauze, etc., can also be used.) As a result of treatment, discomfort such as burning and itching rapidly subsided and remained tolerable for a few hours. Upon the return of burning and itching, the treatment was repeated. As a result of periodic treatments as needed, the symptoms were permanently eliminated typically within 36-58 hours following the treatment initiation (depending on the sensitivity of the individual and the extent of the exposure to the poisonous plant).

### Example 4

Solution A: tris(hydroxymethyl)aminomethane (Tris Base; Trizma®) is dissolved in 70% v/v or higher isopropyl alcohol (“rubbing alcohol”) to obtain a 0.5% solution.

Solution B: 30% (w/w) hydrogen peroxide (H₂O₂ in water).

Freshly harvested leaves of poison oak is minced and rubbed into the selected skin surface of a volunteer subject. This exposure represents an overexposure relative to accidental and unintentional contact with the poisonous plant. After approximately 24 hours the symptoms of UICD are confirmed and the treatment is started. Immediately before application to the affected skin of the volunteer, Solution B. (1 part) is added to Solution A. (9 parts) and thoroughly mixed. An appropriate carrier (i.e., a paper towel) is saturated with the resulting mixture and held tightly to the area showing the symptoms of dermatitis for 20-30 min. (Other carriers, e.g. cotton tissue, gauze, etc., can also be used.) As a result of treatment, discomfort such as burning and itching rapidly subsides and remains tolerable for a few hours. Upon the return of burning and itching, the treatment is repeated. As a result of periodic treatments as needed, the symptoms are permanently eliminated typically within 36-48 hours following treatment initiation (depending on the sensitivity of the individual and the extent of the exposure to the poisonous plant).

Under Examples 1-4. the simplest form of application of the formulations to the affected skin surfaces has been described. It is particularly suitable in health care facilities and in homes. Both Solutions A and B are stable for extended periods in bulk when stored separately from one another according to the manufacturers' recommendations.

In certain cases, Solutions A and B are mixed together well and immediately applied to the skin as described above. Once mixed together the solutions are not stable for any extended period. The potency of peroxide rapidly decreases as soon as after 30-40 min. after preparing the active formulations.

For best results, it is recommended to address the UICD reaction as soon as possible after contact with the urushiol containing plants. Therefore, the most practical methods of application and packaging are in development specifically for use in the remote areas in the wilderness, such as during camping, hunting and fishing trips, military training, etc.

What is claimed is:

1. A method of treating urushiol-induced contact dermatitis in a patient, the method comprising:
identifying a patient in need thereof; and
administering to an affected area of the skin of the patient a composition comprising a pharmaceutically acceptable oxidizing agent; a pharmaceutically acceptable base; and a pharmaceutically acceptable solvent.

2. The method of claim 1, wherein the oxidizing agent is a peroxide of formula R—O—O—H, wherein R is selected from the group consisting of hydrogen, optionally substituted alkyl, and optionally substituted aryl.

3. The method of claim 1, wherein the oxidizing agent is selected from the group consisting of hydrogen peroxide, hydrogen peroxide urea adduct, benzoyl peroxide in pharmaceutically acceptable concentration.

4. The method of claim 1, wherein the base is in the form of a buffer and the buffer is an aqueous buffer.

5. The method of claim 4, wherein the buffer is in the pH range of about 8.5 to about 12.

6. The method of claim 4, wherein the buffer is selected from the group consisting of bicarbonate buffer, carbonate buffer, phosphate buffer, borax, acetate buffer, 2-bis(2-hydroxyethyl)amino-2-(hydroxymethyl)-1,3-propanediol (BIS-TRIS), 1,3-Bis[tris(hydroxymethyl)methylamino]propane (BIS-TRIS Propane), N-(2-hydroxyethyl)piperazine-N′-(2-ethanesulfonic acid) sodium salt (HEPES sodium salt), 4-morpholineethanesulfonic acid sodium salt (MES Sodium Salt), 4-morpholinepropanesulfonic acid sodium salt (MOPS Sodium Salt), and tris(hydroxymethyl)aminomethane (Tris Base; Trizma®).

7. The method of claim 1, wherein the base is a compound of formula R—NH₂, wherein R is selected from the group consisting of hydrogen, optionally substituted alkyl, and optionally substituted aryl.

8. The method of claim 1, wherein the base is water soluble.

9. The method of claim 1, wherein the base is an organic amine selected from the group consisting of triethanolamine, tris(hydroxymethyl)aminomethane, and biologically acceptable esters of naturally occurring amino acids.

10. The method of claim 1, wherein the composition further comprises a pharmaceutically acceptable transdermal transport agent.

11. The method of claim 10, wherein the base and the oxidizing agent are soluble in the transdermal transport agent.

12. The method of claim 10, wherein the transdermal transport agent is selected from the group consisting of ethyl alcohol, isopropyl alcohol, propylene glycol, 2-pyrrolidone, dimethyl sulfoxide, phospholipid liposome, and lecithin organogel.

13. The method of claim 1, the composition further comprises abrasive particles.

14. The method of claim 13, wherein the abrasive particle is selected from the group consisting of various insoluble ground polymers, ground nut shell, ground sand, ground pumice.

15. The composition of claim 1, wherein the composition is aqueous liquid, gel, or foam.

16. The method of claim 1, wherein the administering comprises rubbing the composition on the affected area of the skin of the patient.

17. The method of claim 1, wherein the administering comprises placing an absorbent material comprising the composition on the affected area of the skin of the patient.

18. A device for the treatment of urushiol-induced contact dermatitis, the device comprising:
an absorbent material, contained therein a pharmaceutically acceptable oxidizing agent; a pharmaceutically acceptable base; and a pharmaceutically acceptable solvent;
means for attaching the device to the skin of a patient in need thereof.

19. The device of claim 18, wherein the means for attaching the device comprises an adhesive material, adhesive bands, or adhesive tape.

20. A composition comprising:
a pharmaceutically acceptable oxidizing agent;
a pharmaceutically acceptable base; and
a pharmaceutically acceptable solvent.
